(19) United States
(12) Patent Application Publication
(10) Pub. No.: US 2006/0275228 A1
(43) Pub. Date: Dec. 7, 2006

(54) Skin care compositions containing idebenone

(76) Inventors: Donald Lynn Bissett, Hamilton, OH (US); Larry Richard Robinson, Loveland, OH (US); Shekhar Mitra, Indian Hill, OH (US)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(21) Appl. No.: 11/408,326

(22) Filed: Apr. 21, 2006

Related U.S. Application Data
(60) Provisional application No. 60/678,967, filed on May 9, 2005.

(51) Int. Cl. A61K 31/12; A61K 8/35

(52) U.S. Cl. 424/59; 424/401; 514/690

(57) ABSTRACT

A stable skin care composition, comprising an effective amount of idebenone, at least one additional skin care active, and a dermatologically acceptable carrier. The composition is substantially colorless.

### CROSS REFERENCE TO RELATED APPLICATION

This application claims the benefit of U.S. Provisional Application Ser. No. 60/678,967 filed May 9, 2005.

### FIELD OF THE INVENTION

The present invention relates to skin care compositions, useful for improving the condition and appearance of skin, comprising idebenone and at least one additional skin care active.

### BACKGROUND OF THE INVENTION

The skin is subject to assault by both environmental factors and intrinsic biochemical processes. In particular, oxidative processes cause damage to proteins, lipids, and other cellular components necessary to maintain the skin's health and appearance. This can result in what many consider undesirable “signs of skin aging,” for example, fine lines, wrinkles and uneven skin texture.

Skin care compositions often contain active ingredients to help minimize the signs of skin aging. Idebenone is an effective antioxidant and thus a desirable component in skin care compositions. Applicants have found, however, that compositions containing idebenone at levels previously thought necessary for effectiveness often exhibit discoloration, which consumers may find unacceptable. There exists a need, therefore, to develop skin care compositions containing idebenone that do not exhibit discoloration.

### SUMMARY OF THE INVENTION

The present invention meets the aforementioned need. Applicants believe that the use of idebenone in combination with other skin care actives, in particular with other anti-oxidants, provides an additive and/or synergistic effect. This would allow use of idebenone at lower levels, and result in compositions that provide overall increased skin care benefit, yet which do not exhibit unacceptable discoloration. Therefore, the present invention meets the need of providing consumers with skin care compositions that impart the benefits of idebenone, more effectively improve skin feel and appearance, and which are acceptable to consumers.

According to the first embodiment of the present invention, a stable composition is provided for regulating the condition and appearance of mammalian skin. The composition comprises idebenone, at least one additional skin care active, and a dermatologically acceptable carrier. In addition, the compositions are substantially colorless.

Yet another embodiment provides for depositing the skin care compositions according to the first embodiment onto a substrate, such as a wipe.

Yet another embodiment of the present invention provides a method for regulating the condition of mammalian skin, comprising the step of applying to the skin a stable composition comprising idebenone, at least one additional skin care active, and a dermatologically acceptable carrier, wherein said composition is substantially colorless. The method further may comprise the step of orally ingesting one or more dietary supplements. In addition, the method may comprise the step of applying the composition according to the first embodiment in combination with a delivery enhancement device, such as an iontophoretic delivery system, temperature change element, ultrasound device, spray applicator, and/or energy delivery device.

Yet another embodiment provides for an orally ingestible composition for regulating the condition of mammalian keratinous tissue, comprising idebenone. The composition further may comprise one or more dietary supplements.

Yet another embodiment provides for a kit, comprising a stable skin care composition as described herein.

These and other aspects and advantages of the present invention will become evident to those skilled in the art from a reading of the following detailed description.

### DETAILED DESCRIPTION OF THE INVENTION

Whereas the specification concludes with claims that particularly point out and distinctly claim the present invention, it is believed that the invention will be better understood from the following details.

The present invention describes skin care compositions comprising idebenone, one or more additional skin care actives, and a dermatologically acceptable carrier. The compositions of the present invention may take a variety of final forms, non-limiting examples of which include lotions, creams, emulsions, pastes, milks, liquids, gels, aerosols, solid forms, eye jellies and masks. The compositions may be used in cosmetics, deodorants, antiperspirants, hair care and skin care products, and may be cleansers, moisturizers and combinations thereof. In one embodiment, the compositions are in the form of a lotion or a cream suitable for application to the face, neck and other exposed areas of the body.

The present invention includes both compositions that are intended to be left on the skin indefinitely, or “leave-on” compositions, and compositions which are intended to be removed from skin. Removal may occur through a variety of means, for example wiping or rinsing the skin. The compositions further may be releasably applied to a substrate, suitable for use at a later time. The compositions further may be used in combination with a delivery enhancement device, non-limiting examples of which include an iontophoretic delivery system, temperature change element, ultrasound device, spray applicator, or energy delivery device. The compositions further may be used in conjunction with orally ingestible dietary supplements, including orally ingestible idebenone, to provide enhanced skin care benefits.

Each of the above and additional elements is described herein.

In all embodiments of the present invention, all percentages are by weight of the total composition, unless specifically stated otherwise. All ratios are weight ratios, unless specifically stated otherwise. The number of significant digits conveys neither limitations on the indicated amounts nor on the accuracy of the measurements. All amounts indicating quantities, percentages, proportions, a-values and b-values are understood to be modified by the word “about” unless otherwise specifically indicated. All measurements are understood to be made at about 25° C. and at ambient conditions, where “ambient conditions” means conditions under about one atmosphere of pressure and at about 50% relative humidity.

Herein, “substantially colorless” means that the compositions exhibit minimal discoloration during the course of a reasonable shelf life, and have an a-value, a b-value, or a combination thereof, on the Hunter scale of from about −6 to about +6, alternatively from about −3 to about +3, and alternatively from about −1 to about +1. “Substantially colorless” is understood to include white compositions. The terms “a-value” and “b-value” refer to a value based on the YXZ color system, defined by the Commission Internationale de l'Eclairage (CIE system) to provide a manner of objectively representing perceived color and color differences. X, Y and Z can be expressed in a variety of manners, or “scales,” one of which is the Hunter scale. The Hunter scale has three variables, L, a, and b, which correlate mathematically to X, Y and Z, and is described by Robertson, A. R. in “The CIE 1976 Color Difference Formulas,” Color Research Applications, vol. 2, pp. 7-11 (1977). Samples are analyzed with a MINOLTA® CR-300 Chroma Meter, which generates values for L, a, and b. The value for “a” correlates to a value along the red-green axis, and the value for “b” correlates to a value along the blue-yellow axis. For example, a blue-colored sample will have a negative b-value, whereas a yellow-colored sample will have a positive b-value. A more positive or negative value represents a more intense color.

Herein, “stable” and “stability” mean compositions which are substantially unaltered in chemical state, physical state and/or color. “Stable” further means that the compositions and the skin care actives exhibit stability under reasonable shelf storage conditions and under conditions reasonably expected to be incurred during transport and storage. Transport and storage conditions may include prolonged exposure to temperatures of from about −50° C. to about 60° C. Stability may be determined either by empirical observation or by appropriate methods of chemical analysis that would be known to one of skill in the art.

“Keratinous tissue,” as used herein, means keratin-containing layers disposed as the outermost protective covering of mammals and includes, but is not limited to, skin, hair and nails. “Topical application,” as used herein, means to apply or spread a composition onto the surface of the keratinous tissue.

Herein, “skin care composition” means compositions suitable for topical application on mammalian keratinous tissue. “Skin care actives,” or “actives,” as used herein, means compounds that, when applied to the skin, provide a benefit or improvement to the skin. It is to be understood that skin care actives are useful not only for application to skin, but also to hair, nails and other mammalian keratinous tissue.

Herein, “skin care” means regulating and/or improving skin condition. Herein, “regulating skin condition” means improving skin appearance and/or feel, for example, by providing a smoother appearance and/or feel. Herein, “improving skin condition” means effecting a visually and/or tactilely perceptible positive change in skin appearance and feel. Conditions that may be regulated and/or improved include, but are not limited to, one or more of the following: Reducing the appearance of wrinkles and coarse deep lines, fine lines, crevices, bumps, and large pores; thickening of keratinous tissue (e.g., building the epidermis and/or dermis and/or sub-dermal layers of the skin, and where applicable the keratinous layers of the nail and hair shaft, to reduce skin, hair, or nail atrophy); increasing the convolution of the dermal-epidermal border (also known as the rete ridges); preventing loss of skin or hair elasticity, for example, due to loss, damage and/or inactivation of functional skin elastin, resulting in such conditions as elastosis, sagging, loss of skin or hair recoil from deformation; reduction in cellulite; change in coloration to the skin, hair, or nails, for example, under-eye circles, blotchiness (e.g., uneven red coloration due to, for example, rosacea), sallowness, discoloration caused by telangiectasia or spider vessels, and graying hair.

As used herein, “signs of skin aging,” include, but are not limited to, all outward visibly and tactilely perceptible manifestations, as well as any macro- or microeffects, due to keratinous tissue aging. These signs may result from processes which include, but are not limited to, the development of textural discontinuities such as wrinkles and coarse deep wrinkles, fine lines, skin lines, crevices, bumps, large pores, unevenness or roughness; loss of skin elasticity; discoloration (including undereye circles); blotchiness; sallowness; hyperpigmented skin regions such as age spots and freckles; keratoses; abnormal differentiation; hyperkeratinization; elastosis; collagen breakdown, and other histological changes in the stratum corneum, dermis, epidermis, vascular system (e.g., telangiectasia or spider vessels), and underlying tissues (e.g., fat and/or muscle), especially those proximate to the skin.

“Dermatologically-acceptable,” as used herein, means that the compositions or components thereof so described are suitable for use in contact with mammalian keratinous tissue without undue toxicity, incompatibility, instability, allergic response, and the like.

Herein, “orally acceptable” means that the compositions or components thereof so described are suitable for oral ingestion by a mammal without undue toxicity, incompatibility, instability, allergic response, and the like.

“Effective amount,” as used herein, means an amount of a compound or composition sufficient to significantly induce a positive benefit, including independently or in combination the benefits disclosed herein, but low enough to avoid serious side effects.

Herein, “delivery enhancement device” means any device that increases the amount of active ingredient applied to and/or into the skin relative to the amount of active ingredient that is delivered without using the device.

Herein, “energy delivery device,” means any device used to deliver energy, for example light, heat, sound, electrical and/or magnetic energy, to the skin, hair or other keratinous tissue. Herein, “delivery of energy,” means that surface of the keratinous tissue is exposed to the energy emanating from the energy delivery device, where it may penetrate to the desired layers of the tissue, including the hair shaft and follicle.

Herein, “temperature-change element” means any device used for heating or cooling the composition, the skin, or both. The temperature-change element may provide heat derived from a chemical reaction, heat of solution, crystallisation, an electrical heating element or both. Alternatively, the temperature-change element may be a cooling element and cooling may be provided by an endothermic chemical reaction, an electrical cooling element or both. Alternatively, the temperature change element may comprise a container holding a catalyst and a separate container holding a reactant, wherein the containers are rupturable, so that upon rupture the catalyst and reactant mix to initiate a reaction that induces a temperature change.

Herein, “iontophoretic device” means any device capable of performing iontophoresis. Herein, “iontophoresis” refers to a technique for delivering ions into a person's tissue by placing a solution, or other medium containing the ion, in contact or close proximity with the tissue. The solution or medium containing the ions is typically carried by a first electrode pouch or other suitable receptacle. A second, or dispersive, electrode is placed against the tissue within some proximity of the first electrode. Ions are caused to migrate from the ion-carrying medium through the tissue by the application of an electrical potential or voltage of the appropriate polarity to the two electrodes. A controlled current is established by providing a sufficient voltage differential between the first and second electrodes, and placing a limiting resistance or other current-limiting device elsewhere in the circuit. Examples of iontophoretic devices are described by J. Singh and H. I. Maibach in “Topical iontophoretic drug delivery in vivo: Historical development, devices and future perspectives,” Dermatology, 187(4), pp. 235-38 (1993).

Herein, “dietary supplement” means a dietary ingredient intended to supplement a regular diet, non-limiting examples of which include, vitamins, minerals, herbs or other botanicals, amino acids, enzymes and metabolites. Herein, the dietary supplement is orally acceptable, and is administered orally. Examples of dietary supplements suitable for use in the present invention include, but are not limited to, vitamins, essential fatty acids, and sugar amines. It is to be understood that orally ingestible idebenone also is considered a dietary supplement within the scope of the present invention. The form in which the dietary supplement is administered may vary widely, and includes, for example, tablets, capsules, gel tablets, and liquids. The dietary supplement further may be incorporated into a foodstuff or beverage.

Herein “kit” means a packaging unit comprising at least one composition described herein. The kit may comprise an outer packaging unit, which in turn may comprise one or more inner packaging units. The inner and outer packaging units may be of any type suitable for containing, presenting and reasonably protecting from damage the contents of the kit. The kit may comprise one or more compositions comprising idebenone, one or more orally ingestible dietary supplement, a delivery enhancement device, instructions for use of the device, instructions for complying with suitable application regimens, a substrate, and combinations thereof.

I. Idebenone

The compositions of the present invention comprise effective amounts of idebenone. Herein, “idebenone” means the following compound, its esters and other derivatives, salts, isomers, tautomers, and combinations thereof:

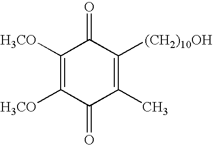

One technical name for idebenone of the present invention is 6-(10-hydroxydecyl)-2,3,-dimethoxy-5-methyl-1,4-benzoquinone. The compositions may comprise from about 0.0001% to about 0.4% idebenone. Alternatively, the compositions may comprise from about 0.001% to about 0.3% idebenone, alternatively from about 0.01% to about 0.2% of idebenone.

Dermatologically acceptable salts include, but are not limited to, alkali metal salts, such as sodium and potassium; alkaline earth metal salts, such as calcium and magnesium; and ammonium and trialkylammonium salts such as trimethylammonium and triethylammonium. Derivatives of idebenone include, but are not limited to, any compounds wherein the CH₃ groups are individually or in combination replaced by amides, esters, amino groups, alkyls, and alcohol esters. Tautomers of idebenone are the isomers of idebenone which can change into one another with ease so that they ordinarily exist in equilibrium. Thus, tautomers of idebenone can be described as having the chemical formula C₁₉H₂₇O₅ and generally having the structure above.

II. Skin Care Actives

The compositions of the present invention comprise at least one additional skin care active, useful for regulating and/or improving the condition of mammalian skin. Classes of suitable skin care actives include, but are not limited to vitamins, peptides and peptide derivatives, sugar amines, sunscreens, oil control agents, particulates, flavonoid compounds, hair growth regulators, non-vitamin antioxidants and/or preservatives, phytosterols, protease inhibitors, tyrosinase inhibitors, anti-inflammatory agents, and mixtures thereof. It should be noted, however, that many skin care actives may provide more than one benefit, or operate via more than one mode of action. Therefore, classifications herein are made for the sake of convenience and are not intended to limit the active to that particular application or applications listed.

A. Vitamins

The compositions of the present invention may comprise one or more vitamins. Herein, “vitamins” means vitamins, pro-vitamins, and their salts, isomers and derivatives. The vitamins may include those which exhibit antioxidant properties, non-limiting examples of suitable vitamins include: vitamin B compounds (including niacinamide, nicotinic acid, C1-C18 nicotinic acid esters, and nicotinyl alcohol; B6 compounds, such as pyroxidine; and B5 compounds, such as panthenol, or “pro-B5”); vitamin A compounds, and all natural and/or synthetic analogs of Vitamin A, including retinoids, carotenoids, and other compounds which possess the biological activity of Vitamin A; vitamin E compounds, or tocopherol, including tocopherol sorbate, tocopherol acetate, other esters of tocopherol; vitamin C compounds, including ascorbyl esters of fatty acids, and ascorbic acid derivatives, for example, ascorbyl glucoside, magnesium ascorbyl phosphate, sodium ascorbyl phosphate, and ascorbyl sorbate. The compositions of the present invention optionally may include vitamins not known to exhibit significant antioxidant properties, for example, vitamin D compounds; vitamin K compounds; and mixtures thereof. In one embodiment, the compositions of the instant invention may comprise from about 0.0001% to about 50%, alternatively from about 0.001% to about 10%, alternatively from about 0.01% to about 5%, and alternatively from about 0.1% to about 1%, of the vitamin.

B. Peptides and Peptide Derivatives

The compositions of the present invention may comprise one or more peptides. Herein, “peptide” refers to peptides containing ten or fewer amino acids, their derivatives, isomers, and complexes with other species such as metal ions (for example, copper, zinc, manganese, and magnesium). As used herein, peptide refers to both naturally occurring and synthesized peptides. In one embodiment, the peptides are di-, tri-, tetra-, penta-, and hexa-peptides, their salts, isomers, derivatives, and mixtures thereof. Examples of useful peptide derivatives include, but are not limited to, peptides derived from soy proteins, palmitoyl-lysine-threonine (pal-KT) and palmitoyl-lysine-threonine-threonine-lysine-serine (pal-KTTKS, available in a composition known as MATRIXYL®), palmitoyl-glycine-glutamine-proline-arginine (pal-GQPR, available in a composition known as RIG®), these three being available from Sederma, France, and Cu-histidine-glycine-glycine (Cu-HGG, also known as IAMIN®).

The compositions may comprise from about 1×10⁻⁷% to about 20%, alternatively from about 1×10⁻⁶% to about 10%, and alternatively from about 1×10⁻⁵% to about 5% of the peptide.

C. Sugar Amines

The compositions of the present invention may comprise a sugar amine, also known as amino sugars, and their salts, isomers, tautomers and derivatives. Sugar amines can be synthetic or natural in origin and can be used as pure compounds or as mixtures of compounds (e.g., extracts from natural sources or mixtures of synthetic materials). For example, glucosamine is generally found in many shellfish and can also be derived from fungal sources. Sugar amine compounds useful in the present invention include, for example, glucosamine sulfate and N-acetyl-D-glucosamine, and also those described in PCT Publication WO 02/076423 and U.S. Pat. No. 6,159,485, issued to Yu, et al. In one embodiment, the composition comprises from about 0.01% to about 15%, alternatively from about 0.1% to about 10%, and alternatively from about 0.5% to about 5%, of the sugar amine.

D. Sunscreens

The compositions of the subject invention may comprise one or more sunscreen actives (or sunscreen agents) and/or ultraviolet light absorbers. Herein, “sunscreen active” includes both sunscreen agents and physical sunblocks. Sunscreen actives and ultraviolet light absorbers may be organic or inorganic. Examples of suitable sunscreen actives and ultraviolet light absorbers are disclosed in The Cosmetic, Toiletry, and Fragrance Association's The International Cosmetic Ingredient Dictionary and Handbook, 10^{th} Ed., Gottschalck, T. E. and McEwen, Jr., Eds. (2004), p. 2267 and pp. 2292-93. Particularly suitable sunscreen agents are 2-ethylhexyl-p-methoxycinnamate (commercially available as PARSOL™ MCX), 4,4′-t-butyl methoxydibenzoyl-methane (commercially available as PARSOL™ 1789), 2-hydroxy-4-methoxybenzophenone, octyldimethyl-p-aminobenzoic acid, digalloyltrioleate, 2,2-dihydroxy-4-methoxybenzophenone, ethyl-4-(bis(hydroxypropyl))aminobenzoate, 2-ethylhexyl-2-cyano-3,3-diphenylacrylate, 2-ethylhexyl-salicylate, glyceryl-p-aminobenzoate, 3,3,5-tri-methylcyclohexylsalicylate, menthyl anthranilate, p-dimethyl-aminobenzoic acid or aminobenzoate, 2-ethylhexyl-p-dimethyl-amino-benzoate, 2-phenylbenzimidazole-5-sulfonic acid, 2-(p-dimethylaminophenyl)-5-sulfonicbenzoxazoic acid, octocrylene, zinc oxide, benzylidene camphor and derivatives thereof, titanium dioxide, and mixtures thereof.

In one embodiment, the composition may comprise from about 1% to about 20%, and alternatively from about 2% to about 10% by weight of the composition, of the sunscreen active and/or ultraviolet light absorber. Exact amounts will vary depending upon the chosen sunscreen active and/or ultraviolet light absorber and the desired Sun Protection Factor (SPF), and are within the knowledge and judgment of one of skill in the art.

E. Oil Control Agents

The compositions of the present invention may comprise one or more compounds useful for regulating the production of skin oil, or sebum, and for improving the appearance of oily skin. Examples of suitable oil control agents include salicylic acid, dehydroacetic acid, benzoyl peroxide, vitamin B3 compounds (for example, niacinamide), their isomers, esters, salts and derivatives, and mixtures thereof. The compositions may comprise from about 0.0001% to about 15%, alternatively from about 0.01% to about 10%, alternatively from about 0.1% to about 5%, and alternatively from about 0.2% to about 2%, of an oil control agent.

F. Particulates

The compositions of the present invention may comprise one or more particulate materials. Nonlimiting examples of particulate materials useful in the present invention include colored and uncolored pigments, interference pigments (nonlimiting examples include mica, layered with about 50-300 nm films of TiO₂, Fe₂O₃ silica, tin oxide, Cr₂O₃, and mixtures thereof; spherical TiO₂ particles having a size of from about 100 to about 300 nanometers; or alternatively, spherical TiO₂ particles having a size of from about 1 to about 30 micrometers; and mixtures thereof), inorganic powders (for example, iron oxides, ferric ammonium ferrocyanide, manganese violet, ultramarine blue, and chrome oxide), organic powders (for example, phthalocyanine blue and green pigment), composite powders, optical brightener particles, and combinations thereof. These particulates can, for instance, be platelet shaped, spherical, elongated or needle-shaped, or irregularly shaped; surface coated or uncoated; porous or non-porous; charged or uncharged; and can be added to the current compositions as a powder or as a pre-dispersion.

In one embodiment, the compositions may comprise from about 0.01% to about 20%, alternatively from about 0.05% to about 10%, alternatively from about 0.1% to about 5%, of particulate materials.

G. Flavonoids

The compositions of the present invention may comprise a flavonoid. The flavonoid can be synthetic materials or obtained as extracts from natural sources, which also further may be derivatized. Examples of classes of suitable flavonoids are disclosed in U.S. Pat. No. 6,235,773, issued to Bissett, and include, but are not limited to, unsubstituted flavanone, methoxy flavanones, unsubstituted chalcone, 2′,4-dihydroxy chalcone, and mixtures thereof. In one embodiment, the flavonoids are unsubstituted flavanones, unsubstituted chalcone (especially the trans-isomer), their glucosyl derivatives, and mixtures thereof. Other examples of suitable flavonoids include flavanones such as hesperidin and glucosyl hesperidin, isoflavones such as soy isoflavones, including but not limited to genistein, daidzein, and equol, their glucosyl derivatives, and mixtures thereof.

The compositions of the present invention may comprise from about 0.01% to about 20%, alternatively from about 0.1% to about 10%, and alternatively from about 0.5% to about 5% of flavonoids.

H. Hair Growth Regulators

The compositions of the present invention may comprise compounds useful for regulating hair growth. Suitable hair growth regulators include, but are not limited to, hexamidine, butylated hydroxytoluene (BHT), hexanediol, panthenol and pantothenic acid derivates, their isomers, salts and derivatives, and mixtures thereof. The compositions of the present invention may comprise from about 0.0001% to about 20%, alternatively from about 0.001% to about 10%, alternatively from about 0.01% to about 5%, and alternatively from about 0.1% to about 2% of hair growth regulators.

I. Other Skin Care Actives

The compositions of the present invention further may comprise non-vitamin antioxidants, preservatives, phytosterols and/or plant hormones, protease inhibitors, extracts, tyrosinase inhibitors, anti-inflammatory agents and N-acyl amino acid compounds.

Suitable non-vitamin antioxidants include, but are not limited to, BHT (butylated hydroxy toluene), L-ergothioneine (available as THIOTANE™); tetrahydrocurcumin, cetyl pyridinium chloride, camosine, diethylhexyl syrinylidene malonate (available as OXYNEX™), ubiquinone (co-enzyme Q10), and combinations thereof.

Suitable examples of plant sterols and/or plant hormones include, but are not limited to, sitosterol, stigmasterol, campesterol, brassicasterol, kinetin, zeatin, and mixtures thereof.

Suitable protease inhibitors include, but are not limited to, hexamidine, vanillin acetate, menthyl anthranilate, and mixtures thereof.

Suitable extracts include yeast extract such as the yeast culture extract Piteras.

Suitable tyrosinase inhibitors include, but are not limited to, sinablanca (mustard seed extract), tetrahydrocurcumin, cetyl pyridinium chloride, and mixtures thereof.

Suitable anti-inflammatory agents include, but are not limited to, glycyrrhizic acid (also known as glycyrrhizin, glycyrrhixinic acid, and glycyrrhetinic acid glycoside), glycyrrhetenic acid, and combinations thereof.

Suitable N-acyl amino acid compounds include, but are not limited to, N-acyl phenylalanine, N-acyl tyrosine, their isomers, including their D and L isomers, salts, derivatives, and mixtures thereof. An example of a suitable N-acyl amino acid is N-undecylenoyl-L-phenylalanine is commercially available under the tradename SEPIWHITE® from Seppic (France).

Other useful skin care actives include dehydroepiandrosterone (DHEA), its analogs and derivatives; alpha- and beta-hydroxyacids, including glycolic acid and octanoyl salicylate, arbutin, dimethyl aminoethanol (DMAE), kojic acid, dihydroxy acetone (DHA), soy proteins and peptides (for example, protease inhibitors such as soybean trypsin inhibitor, and Bowman-Birk inhibitor), arbutin, their isomers, salts, and derivatives, and mixtures thereof.

III. Dermatologically Acceptable Carrier

The topical compositions of the present invention also may comprise a dermatologically acceptable carrier. Herein, “dermatologically acceptable carrier” means that the carrier is suitable for topical application to the keratinous tissue, has good aesthetic properties, is compatible with the actives of the present invention and any other components, and will not cause unduesafety or toxicity concerns. The compositions of the present invention typically comprise from about 50% to about 99.99% of the dermatologically acceptable carrier, alternatively from about 60% to about 99.9% of the carrier, alternatively from about 70% to about 98% of the carrier, and alternatively from about 80% to about 95% of the carrier.

The dermatologically acceptable carrier can be in a wide variety of forms. Non-limiting examples include simple solutions (water-based or oil-based), solid forms (for example, gels or sticks) and emulsions. Herein, “emulsions” generally contain an aqueous phase and a lipid or oil. Lipids and oils may be derived from animals, plants, or petroleum and may be natural or synthetic. Emulsion carriers include, but are not limited to, oil-in-water, water-in-oil, silicone-in-water, water-in-silicone, water-in-oil-in-water, and oil-in-water-in-silicone emulsions. In one embodiment, the dermatologically acceptable carrier comprises oil-in-water emulsions and water-in-oil emulsions. In yet another embodiment, the dermatologically acceptable carrier is an oil-in-water emulsion.

Emulsion

The compositions of the present invention may be in the form of an emulsion Emulsions may contain a humectant, for example, glycerin. Emulsions further may comprise an emulsifier. Emulsifiers may be nonionic, anionic or cationic. Suitable emulsifiers are disclosed in, for example, U.S. Pat. No. 3,755,560 issued to Dickert et al., U.S. Pat. No. 4,421,769, issued to Dixon et al., and McCutcheon's Detergents and Emulsifiers, North American Edition, pages 317-324 (1986). Suitable emulsions may have a wide range of viscosities, depending on the desired product form.

IV. Optional Ingredients

A. Surfactants

The compositions of the present invention may comprise one or more surfactants. These surfactants or combinations of surfactants should be mild, which means that these surfactants provide sufficient cleansing or detersive benefits but do not overly dry the skin. Surfactants useful herein include those selected from the group consisting of anionic surfactants, amphoteric surfactants, zwitterionic surfactants, cationic surfactants, nonionic surfactants and mixtures thereof. Examples of such surfactants are found in and U.S. Pat. No. 5,624,666, issued to Coffindaffer et al. Concentrations of these surfactant are from about 0.1% to about 20%, alternatively from about 0.5% to about 15%, and alternatively from about 1% to about 10%.

B. Other Particulate Materials

The present invention may comprise from about 0.1% to about 30%, alternatively from about 0.5% to about 15%, and alternatively from about 1% to about 5%, of particulate materials, including cleansing and exfoliating agents. The particulate cleansing or exfoliating agents can be derived from a wide variety of materials including those derived from inorganic, organic, natural, and synthetic sources. Non-limiting examples of these materials include almond meal, alumina, aluminum oxide, aluminum silicate, apricot seed powder, attapulgite, barley flour, bismuth oxychloride, boron nitride, calcium carbonate, calcium phosphate, calcium pyrophosphate, calcium sulfate, cellulose, chalk, chitin, clay, corn cob meal, corn cob powder, corn flour, corn meal, corn starch, diatomaceous earth, dicalcium phosphate, dicalcium phosphate dihydrate, fuller's earth, hydrated silica, hydroxyapatite, iron oxide, jojoba seed powder, kaolin, loofah, magnesium trisilicate, mica, microcrystalline cellulose, montmorillonite, oat bran, oat flour, oatmeal, peach pit powder, pecan shell powder, polybutylene, polyethylene, polyisobutylene, polymethylstyrene, polypropylene, polystyrene, polyurethane, nylon, TEFLON® (polytetrafluoroethylene), polyhalogenated olefins, pumice rice bran, rye flour, sericite, silica, silk, sodium bicarbonate, sodium silicoaluminate, soy flour synthetic hectorite, talc, tin oxide, titanium dioxide, tricalcium phosphate, walnut shell powder, wheat bran, wheat flour, wheat starch, zirconium silicate, and mixtures thereof. Also useful are particles made from mixed polymers (e.g., copolymers, terpolymers, etc.), among such are polyethylene/polypropylene copolymer, polyethylene/propylene/isobutylene copolymer, polyethylene/styrene copolymer, and mixtures thereof. Typically, the polymeric and mixed polymeric particles are treated via an oxidation process to destroy, for example, impurities. The polymeric and mixed polymeric particles can also optionally be cross linked with a variety of common crosslinking agents, non-limiting examples including butadiene, divinyl benzene, methylenebisacrylamide, allyl ethers of sucrose, allyl ethers of pentaerythritol, and mixtures thereof. Other examples of useful particles include waxes and resins such as paraffins, carnuba wax, ozekerite wax, candellila wax, and urea-formaldehyde resins. When such waxes and resins are used herein it is important that these materials are solids at ambient skin temperatures.

C. Conditioning Agents

The compositions of the present invention may comprise from about 0.1% to about 50%, alternatively from about 0.5% to about 30%, alternatively from about 1% to about 20%, alternatively from about 2% to 15%, of a conditioning agent. These conditioning agents include, but are not limited to, hydrocarbon oils and waxes, silicones, fatty acid derivatives, cholesterol, cholesterol derivatives, diglycerides, triglycerides, vegetable oils, vegetable oil derivatives, acetoglyceride esters, alkyl esters, alkenyl esters, lanolin, wax esters, beeswax derivatives, sterols and phospholipids, salts, isomers and derivatives thereof, and combinations thereof.

Non-limiting examples of hydrocarbon oils and waxes suitable for use herein include petrolatum, mineral oil, micro-crystalline waxes, polyalkenes, paraffins, cerasin, ozokerite, polyethylene, perhydrosqualene, polyalphaolefins, hydrogenated polyisobutenes and combinations thereof.

Non-limiting examples of silicone oils suitable for use herein include dimethicone copolyol, dimethylpolysiloxane, diethylpolysiloxane, mixed C₁₋₃₀ alkyl polysiloxanes, phenyl dimethicone, dimethiconol, and combinations thereof. In one embodiment, the silicone oils are non-volatile silicone oils selected from the group consisting of dimethicone, dimethiconol, mixed C₁₋₃₀ alkyl polysiloxanes, silicone crosspolymers, and combinations thereof. These and other examples of silicone oils useful herein are described in U.S. Pat. No. 5,011,681, issued to Ciotti et al.

Non-limiting examples of silicone cross-polymers suitable for use herein include acrylate/bis-hydroxypropyl dimethicone crosspolymer, C₃₀₋₄₅ alkyl cetearyl dimethicone crosspolymer, acrylate/bis-hydroxypropyl dimethicone crosspolymer, C₃₀₋₄₅ alkyl cetearyl dimethicone crosspolymer, cetearyl dimethicone/vinyl dimethicone crosspolymer, dimethicone crosspolymer, dimethicone crosspolymer-3, dimethicone/phenyl vinyl dimethicone crosspolymer, dimethicone/vinyl dimethicone crosspolymer, diphenyl dimethicone crosspolymer, divinyldimethicone/dimethicone crosspolymer, polyethylene glycol (PEG)-10 dimethicone crosspolymer, PEG-12 dimethicone crosspolymer, PEG-10 dimethicone/vinyl dimethicone crosspolymer, PEG-10/lauryl dimethicone crosspolymer, PEG-15/lauryl dimethicone crosspolymer, trifluoropropyl dimethicone/trifluoropropyl divinyldimethicone crosspolymer, vinyl dimethicone/lauryl dimethicone crosspolymer, vinyldimethyl/trimethylsiloxysilicate stearyl dimethicone crosspolymer, polysilicone-11, and mixtures thereof.

Also useful herein are various C₁₋₃₀ monoesters and polyesters of sugars and related materials, for example, sucrose esters of fatty acids (SEFA).

A variety of emollients may be employed as conditioning agents. These emollients may be selected from one or more of the following classes: triglyceride esters acetoglyceride esters, alkyl esters of fatty acids having 10 to 20 carbon atoms, alkenyl esters of fatty acids having 10 to 20 carbon atoms, fatty acids having 10 to 20 carbon atoms, fatty alcohols having 10 to 20 carbon atoms, lanolin, polyhydric alcohol esters, wax esters, vegetable waxes, phospholipids, sterols, amides, isomers, salts, derivatives and mixtures thereof.

These and other suitable conditioning agents are exemplified in U.S. Pat. No. 5,997,890, issued to Sine et al.

D. Structuring Agent

The compositions of the present invention may contain a structuring agent. Structuring agents are especially suitable in the emulsions of the present invention, for example, in the oil-in-water emulsions of the present invention. Without being limited by theory, it is believed that the structuring agent assists in providing rheological characteristics (for example yield and structural characteristics) to the composition which contribute to the stability of the composition. When present, the compositions of the present invention comprise from about 0.1% to about 20%, alternatively from about 0.5% to about 10%, and alternatively from about 1% to about 5%, of one or more structuring agents.

The structuring agents of the present invention may be selected from the group consisting of stearic acid, palmitic acid, stearyl alcohol, cetyl alcohol, behenyl alcohol, palmitic acid, the polyethylene glycol ether of stearyl alcohol having an average of from about 1 to about 5 ethylene oxide units, the polyethylene glycol ether of cetyl alcohol having an average of from about 1 to about 5 ethylene oxide units, and mixtures thereof. In one embodiment, structuring agents of the present invention are selected from the group consisting of stearyl alcohol, cetyl alcohol, behenyl alcohol, the polyethylene glycol ether of stearyl alcohol having an average of about 2 ethylene oxide units (steareth-2), the polyethylene glycol ether of cetyl alcohol having an average of about 2 ethylene oxide units, and mixtures thereof. In another embodiment, structuring agents are selected from the group consisting of stearic acid, palmitic acid, stearyl alcohol, cetyl alcohol, behenyl alcohol, steareth-2, and mixtures thereof.

E. Thickening Agent

The compositions of the present invention may comprise from about 0.1% to about 5%, alternatively from about 0.1% to about 4%, and alternatively from about 0.25% to about 3%, of one or more thickening agents, including thickeners and gelling agents. Nonlimiting classes of thickening agents include crosslinked polyacrylate polymers, polyacrylamide polymers, polysaccharides and gums. In one embodiment, compositions of the present invention include a thickening agent selected from carboxylic acid polymers, crosslinked polyacrylate polymers, polyacrylamide polymers, and mixtures thereof. In yet another embodiment, the thickening agent is selected from carboxylic acid polymers, polyacrylamide polymers, and mixtures thereof.

F. Substrates

The compositions of the present invention can be applied directly to the skin. Additionally or alternatively, the compositions can be applied with the use of a suitable applicator comprising a substrate material. In one embodiment, the composition is applied to the substrate such that the substrate releasably holds the composition. The compositions of the present invention are suitable for use in combination with a substrate to effect personal cleansing, skin treatment, or other personal care uses. In one embodiment, the composition is pre-combined with or deposited onto the substrate to form a wipe product, non-limiting examples of which include disposable wipe products, masks and eye jellies. Herein, “wipe product” means a substrate and a composition of the present invention which are pre-combined for later use. Wipe products may be packaged in a relatively dry state, and wetted prior to use, or may be packaged having already been wetted.

Suitable wipe substrates include, but are not limited to, nonwovens, films, foams, sponges, and combinations thereof. In one embodiment, wipe substrates comprise a porous material which is capable of holding the composition within the pores of the substrate. Therefore, in one embodiment, the substrate is a nonwoven.

Techniques for combining wipe substrates with a cleansing or treating composition, and for their packaging, are well known in the art and are applicable to the present invention. In general, the wipe substrate is combined with the composition by one or more techniques involving coating, immersing, dipping, spraying, extruding. In general, the wipes are combined with an amount of the composition sufficient to provide effective skin application. In one embodiment, the product may be combined with the substrate in amounts of from about 0.1 gram of lotion per gram of substrate to about 10 grams of lotion per gram of substrate.

V. Method

The present invention provides for a method for regulating the condition of mammalian skin. Regulating skin condition means improving skin appearance and/or feel, for example, providing a smoother, more even appearance and/or feel, as described further herein.

The method of regulating skin conditions comprises the step of topically applying to the skin and/or other keratinous tissue an effective amount of a skin care composition of the present invention. Any part of the external portion of the skin can be treated. The amount of the composition applied, the frequency of application and the period of use will vary widely depending upon the level of components of a given composition and the level of regulation desired. For example, from about 0.01 g composition/cm² to about 1 g composition/cm² of keratinous tissue may be applied. In one embodiment, the compositions are applied at least once daily, where “daily” and “days” mean a 24-hour period. For example, the compositions may be applied daily for 30 consecutive days, alternatively for 14 consecutive days, alternatively for 7 consecutive days and alternatively for 2 consecutive days.

The application of the present compositions may be done using the palms of the hands and/or fingers, or by using an implement (e.g., a cotton ball, swab, pad, substrate, etc.). Where the composition has been applied to a substrate, the application is by means of wiping, dabbing, scrubbing, or other suitable means, the skin or keratinous tissue with the substrate. Depending upon the form of the composition, the substrate containing the composition may be wetted prior to application.

The method may comprise the step of inducing a temperature change in the composition either simultaneously or sequentially with the step of applying the composition to the keratinous tissue. Alternatively, the method may comprise the step of inducing a temperature change in the keratinous tissue either simultaneously or sequentially with application of the composition.

In one embodiment, regulating skin condition is practiced by topically applying a composition in the form of a lotion, cleansing milk, cream, gel, foam, ointment, paste, emulsion, tonic, cosmetic, or the like and by leaving said composition on the skin or other keratinous tissue to produce some aesthetic, prophylactic, therapeutic or other benefit (i.e., a “leave-on” composition). In an alternative embodiment, for example as with a cleansing milk, the composition may be rinsed, wiped, or otherwise removed from the skin or keratinous tissue after application.

In yet another embodiment, regulating skin condition is practiced by orally ingesting one or more dietary supplements in conjunction with the topical application of the compositions described herein. The dietary supplement may comprise orally ingestible idebenone. Alternatively, ingestion of idebenone may occur independently of topical administration of the composition.

In yet another embodiment, the compositions are applied with a delivery enhancement device, non-limiting examples of which include an iontophoretic delivery system, one or more temperature change elements, an ultrasound device, spray applicator, and/or an energy delivery device.

The present invention further may comprise a kit, said kit comprising an outer packaging unit, which in turn may comprise one or more smaller, inner packaging units. The inner packaging units may comprise one or more of the individual components of the kit. The inner and outer packaging units may be of any type suitable for containing, presenting and/or reasonably protecting from damage the contents of the kit. The kit may comprise one or more compositions comprising idebenone, one or more orally ingestible dietary supplements, a delivery enhancement device, instructions for use of the device, instructions for complying with suitable application regimens, a substrate, and combinations thereof. The compositions and/or the orally ingestible dietary supplements may be packaged in quantities suitable for use in a single application regimen, and alternatively in quantities suitable for multiple application regimens.

### EXAMPLES

The following are examples of skin care compositions according to the present invention.

| | Content in formulation | | | | | |
|---|---|---|---|---|---|---|
| | (g component per 100 g formulation) | | | | | |
| Component | A | B | C | D | E | F |
| Phase A | | | | | | |
| Water (to 100 g) | q.s. to | q.s. to | q.s. to | q.s. to | q.s. to | q.s. to |
| | 100 | 100 | 100 | 100 | 100 | 100 |
| Disodium EDTA | 0.100 | 0.10 | 0.10 | 0.10 | 0.10 | 0.10 |
| Idebenone | 0.0001 | 0.001 | 0.01 | 0.1 | 0.15 | 0.20 |
| Niacinamide | 5.0 | 4.0 | 2.0 | 1.0 | 0.001 | 0.01 |
| Peptide¹ | 0.0001 | 0 | 0 | 0 | 0 | 0 |
| Sugar Amine¹ | 0 | 0.01 | 0 | 0 | 0 | 0 |
| N-acyl amino | 0 | 0 | 0.01 | 0 | 0 | 0 |
| acid¹ | | | | | | |
| Hair growth | 0 | 0 | 0 | 0.1 | 0 | 0 |
| regulator¹ | | | | | | |
| Sunscreen¹ | 0 | 0 | 0 | 0 | 10 | 0 |
| Oil Control | 0 | 0 | 0 | 0 | 0 | 1.0 |
| Agent¹ | | | | | | |
| Particulate¹ | 0 | 0 | 0 | 0 | 0 | 0 |
| Flavonoid¹ | 0 | 0 | 0 | 0 | 0 | 0 |
| Tyrosinase | 0 | 0 | 0 | 0 | 0 | 0 |
| Inhibitor¹ | | | | | | |
| Plant Sterol¹ | 0 | 0 | 0 | 0 | 0 | 0 |
| Protease | 0 | 0 | 0 | 0 | 0 | 0 |
| Inhibitor¹ | | | | | | |
| Anti- | 0 | 0 | 0 | 0 | 0 | 0 |
| inflammatory¹ | | | | | | |
| Citric acid | 1.500 | 0 | 0 | 0 | 0 | 0 |
| Polyquaternium | 0 | 0 | 0 | 0 | 0 | 0 |
| 37 | | | | | | |
| Phase B | | | | | | |
| Isohexadecane | 3.000 | 3.000 | 0 | 0 | 3.00 | 3.00 |
| Isopropyl | 1.330 | 1.330 | 0 | 0 | 1.33 | 1.33 |
| isostearate | | | | | | |
| Isopropyl N- | 0 | 0 | 5.00 | 6.00 | 0 | 0 |
| laurosylsarcosinate | | | | | | |
| Sucrose | 0.670 | 0.670 | 0 | 0 | 0.67 | 0.67 |
| polycottonseedate | | | | | | |
| Polymethylsilsesquioxane | 0.250 | 0.250 | 0.25 | 0.25 | 0.25 | 0.25 |
| Cetearyl glucoside + cetearyl | 0.200 | 0.200 | 0.20 | 0.20 | 0.20 | 0.20 |
| alcohol | | | | | | |
| Behenyl alcohol | 0.400 | 0.400 | 0.40 | 0.40 | 0.40 | 0.40 |
| Ethylparaben | 0.200 | 0.200 | 0.20 | 0.20 | 0.20 | 0.20 |
| Propylparaben | 0.100 | 0.100 | 0.10 | 0.10 | 0.10 | 0.10 |
| Cetyl alcohol | 0.320 | 0.320 | 0.32 | 0.32 | 0.32 | 0.32 |
| Stearyl alcohol | 0.480 | 0.480 | 0.48 | 0.48 | 0.48 | 0.48 |
| Tocopheryl | 0.500 | 0.500 | 0.50 | 0.50 | 0.50 | 0.50 |
| acetate | | | | | | |
| PEG-100 stearate | 0.100 | 0.100 | 0.10 | 0.10 | 0.10 | 0.10 |
| Glycerin | 7.000 | 7.000 | 7.00 | 7.00 | 7.00 | 7.00 |
| Titanium dioxide | 0.604 | 0.604 | 0.60 | 0.60 | 0.60 | 0.60 |
| Phase C | | | | | | |
| Polyacrylamide + C13-14 | 3.000 | 2.000 | 2.00 | 2.00 | 2.00 | 2.00 |
| isoparaffin + laureth-7 | | | | | | |
| Panthenol | 1.000 | 1.000 | 1.00 | 1.00 | 1.00 | 1.00 |
| Benzyl alcohol | 0.400 | 0.400 | 0.40 | 0.40 | 0.40 | 0.40 |
| Phase D | | | | | | |
| Dimethicone + dimethiconol | 2.000 | 2.000 | 2.00 | 2.00 | 2.00 | 2.00 |
| TOTAL | 100 | 100 | 100 | 100 | 100 | 100 |
| | Content in formulation | | | | | |
| | (g component per 100 g formulation) | | | | | |
| Component | G | H | I | J | K | L |
| Phase A | | | | | | |
| Water (to 100 g) | q.s. to | q.s. to | q.s. to | q.s. to | q.s. to | q.s. to |
| | 100 | 100 | 100 | 100 | 100 | 100 |
| Disodium EDTA | 0.10 | 0.10 | 0.10 | 0.10 | 0.10 | 0.100 |
| Idebenone | 0.25 | 0.30 | 0.35 | 0.4 | 0.4 | 0.0001 |
| Niacinamide | 0.1 | 1.0 | 2.0 | 3.0 | 4.0 | 5.0 |
| Peptide¹ | 0 | 0 | 0 | 0 | 0 | 0 |
| Sugar Amine¹ | 0 | 0 | 0 | 0 | 0 | 0 |
| N-acyl amino | 0 | 0 | 0 | 0 | 0 | 0 |
| acid¹ | | | | | | |
| Hair growth | 0 | 0 | 0 | 0 | 0 | 0 |
| regulator¹ | | | | | | |
| Sunscreen¹ | 0 | 0 | 0 | 0 | 0 | 0 |
| Oil Control | 0 | 0 | 0 | 0 | 0 | 0 |
| Agent¹ | | | | | | |
| Particulate¹ | 1.0 | 0 | 0 | 0 | 0 | 0 |
| Flavonoid¹ | 0 | 0.1 | 0 | 0 | 0 | 0 |
| Tyrosinase | 0 | 0 | 0.1 | 0 | 0 | 0 |
| Inhibitor¹ | | | | | | |
| Plant Sterol¹ | 0 | 0 | 0 | 0.1 | 0 | 0 |
| Protease | 0 | 0 | 0 | 0 | 0.1 | 0 |
| Inhibitor¹ | | | | | | |
| Anti- | 0 | 0 | 0 | 0 | 0 | 0.1 |
| inflammatory¹ | | | | | | |
| Citric acid | 0 | 0 | 0 | 0 | 0 | 0 |
| Polyquaternium | 0 | 0 | 1.500 | 0 | 0 | 0 |
| 37 | | | | | | |
| Phase B | | | | | | |
| Isohexadecane | 3.00 | 3.00 | 3.00 | 3.00 | 3.00 | 3.000 |
| Isopropyl | 1.33 | 1.33 | 1.33 | 1.33 | 1.33 | 1.330 |
| isostearate | | | | | | |
| Isopropyl N- | 0 | 0 | 0 | 0 | 0 | 0 |
| laurosylsarcosinate | | | | | | |
| Sucrose | 0.67 | 0.67 | 0.67 | 0.67 | 0.67 | 0.670 |
| polycottonseedate | | | | | | |
| Polymethylsilsesquioxane | 0.25 | 0.25 | 0.25 | 0.25 | 0.25 | 0.250 |
| Cetearyl glucoside + cetearyl | 0.20 | 0.20 | 0.20 | 0.20 | 0.20 | 0.200 |
| alcohol | | | | | | |
| Behenyl alcohol | 0.40 | 0.40 | 0.40 | 0.40 | 0.40 | 0.400 |
| Ethylparaben | 0.20 | 0.20 | 0.20 | 0.20 | 0.20 | 0.200 |
| Propylparaben | 0.10 | 0.10 | 0.10 | 0.10 | 0.10 | 0.100 |
| Cetyl alcohol | 0.32 | 0.32 | 0.32 | 0.32 | 0.32 | 0.320 |
| Stearyl alcohol | 0.48 | 0.48 | 0.48 | 0.48 | 0.48 | 0.480 |
| Tocopheryl | 0.50 | 0.50 | 0.50 | 0.50 | 0.50 | 0.500 |
| acetate | | | | | | |
| PEG-100 stearate | 0.10 | 0.10 | 0.10 | 0.10 | 0.10 | 0.100 |
| Glycerin | 7.00 | 7.00 | 7.00 | 7.00 | 7.00 | 7.000 |
| Titanium dioxide | 0.60 | 0.60 | 0.60 | 0.60 | 0.60 | 0.60 |
| Phase C | | | | | | |
| Polyacrylamide + C13-14 | 2.00 | 2.00 | 0 | 2.00 | 2.00 | 2.000 |
| isoparaffin + laureth-7 | | | | | | |
| Panthenol | 1.00 | 1.00 | 1.00 | 1.00 | 1.00 | 1.000 |
| Benzyl alcohol | 0.40 | 0.40 | 0.40 | 0.40 | 0.40 | 0.400 |
| Phase D | | | | | | |
| Dimethicone + dimethiconol | 2.00 | 2.00 | 2.00 | 2.00 | 2.00 | 2.000 |
| TOTAL | 100 | 100 | 100 | 100 | 100 | 100 |

¹It is understood that this ingredient may comprise one or more of the disclosed examples of this class of compounds in their respective disclosed percentages.

In a suitable vessel, combine the Phase A ingredients and heat to 75° C. In a separate suitable vessel, combine Phase B ingredients and heat to 75° C. Next, add Phase A to Phase B and mill the resulting emulsion (e.g., with a TEKMAR® T-25). Then, add Phase C to the emulsion and cool the emulsion to 45° C. while stirring. At 45° C., add the remaining ingredients. Cool the product and stir to 30° C. and pour into suitable containers.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A stable skin care composition, comprising:
a) an effective amount of idebenone;
b) at least one additional skin care active; and
c) a dermatologically acceptable carrier,
wherein said skin care composition is substantially colorless.

2. A skin care composition according to claim 1, wherein said composition comprises from about 0.0001% to about 0.4% of idebenone.

3. A skin care composition according to claim 1, wherein said skin care active is selected from the group consisting of vitamin A compounds, vitamin B compounds, vitamin C compounds, vitamin E compounds, alpha-hydroxyacids, beta-hydroxyacids, peptides, sunscreen agents, ultraviolet light absorbers, N-acyl amino acid compounds, anti-inflammatory compounds, non-vitamin antioxidants, yeast extract, yeast culture extracts, preservatives and mixtures thereof.

4. A skin care composition according to claim 3, wherein said vitamin A compound is retinyl propionate.

5. A skin care composition according to claim 3, wherein said vitamin B compound is selected from the group consisting of niacinamide, nicotinic acid, C1-C18 nicotinic acid esters, nicotinyl alcohol, pyroxidine, panthenol, and mixtures thereof.

6. A skin care composition according to claim 5, wherein said vitamin B compound is selected from the group consisting of niacinamide, panthenol, and mixtures thereof.

7. A skin care composition according to claim 3, wherein said vitamin E compound is selected from the group consisting of tocopherol sorbate, tocopherol acetate, esters of tocopherol, and mixtures thereof.

8. A skin care composition according to claim 3, wherein said vitamin C compound is selected from the group consisting of ascorbyl esters of fatty acids, ascorbyl glucoside, magnesium ascorbyl phosphate, sodium ascorbyl phosphate, ascorbyl sorbate, and mixtures thereof.

9. A skin care composition according to claim 3, wherein said peptide is selected from the group consisting of palmitoyl-lysine-threonine, palmitoyl-lysine-threonine-threonine-lysine-serine, Cu-histidine-glycine-glycine, palmitoyl-glycine-glutamine-proline-arginine, camosine, and mixtures thereof.

10. A skin care composition according to claim 1, wherein said composition is deposited onto a substrate.

11. A skin care composition according to claim 1, wherein said composition has an a-value of from about −3 to about +3 on the Hunter scale, a b-value of from about −3 to about +3 on the Hunter scale, and combinations thereof.

12. A skin care composition according to claim 11, wherein said composition has an a-value of from about −1 to about +1 on the Hunter scale, a b-value of from about −1 to about +1 on the Hunter scale, and combinations thereof.

13. A stable skin care composition, comprising:
a) from about 0.0001% to about 0.4% of idebenone;
b) at least one additional skin care active, selected from the group consisting of vitamin A compounds, vitamin B compounds, vitamin C compounds, vitamin E compounds, alpha-hydroxyacids, beta-hydroxyacids, peptides, sunscreens, ultraviolet light absorbers, N-acyl amino acid compounds, anti-inflammatory compounds, non-vitamin antioxidants, preservatives, and mixtures thereof;
c) a dermatologically acceptable carrier; and
wherein said skin care composition has an a-value of from about −3 to about +3 on the Hunter scale, a b-value of from about −3 to about +3 on the Hunter scale, and combinations thereof.

14. A skin care composition according to claim 13, wherein said composition has an a-value of from about −1 to about +1 on the Hunter scale, a b-value of from about −1 to about +1 on the Hunter scale, and combinations thereof.

15. A skin care composition according to claim 13, wherein said composition comprises from about 0.01% to about 0.2% of idebenone.

16. A skin care composition according to claim 13, wherein said skin care active is selected from the group consisting of vitamin B compounds, vitamin C compounds, vitamin E compounds, sunscreens, ultraviolet light absorbers, and mixtures thereof.

17. A skin care composition according to claim 16, wherein said skin care active is selected from the group consisting of niacinamide, ascorbyl glucoside, TiO₂ and mixtures thereof.

18. A skin care composition according to claim 13, wherein said composition is deposited onto a substrate.

19. An orally ingestible composition for regulating the condition of mammalian keratinous tissue comprising:
a. an effective amount of idebenone;
b. an orally acceptable carrier.

20. The composition of claim 19, said composition further comprising at least one additional dietary supplement.

21. A method for regulating the condition of mammalian keratinous tissue, comprising the step of applying a stable skin care composition to the keratinous tissue, said composition comprising:
a) an effective amount of idebenone;
b) at least one additional skin care active; and
c) a dermatologically acceptable carrier;
wherein said skin care composition is substantially colorless.

22. The method of claim 21, further comprising the step of orally ingesting a dietary supplement.

23. The method of claim 22, wherein said dietary supplement is idebenone.

24. The method of claim 21, further comprising the step of applying the skin care composition to the keratinous tissue in combination with a delivery enhancement device.

25. The method of claim 24, wherein said delivery enhancement device is selected from the group consisting of an iontophoretic delivery system, a temperature change element, a spray applicator, an energy delivery device, and combinations thereof.

26. A kit comprising a stable skin care composition, said composition further comprising:
a) an effective amount of idebenone;
b) at least one additional skin care active; and
c) a dermatologically acceptable carrier.

27. The kit of claim 26, said kit further comprising a dietary supplement.

28. The kit of claim 27, said kit further comprising a delivery enhancement device.

29. The kit of claim 28, said kit further comprising a substrate.
